(19) United States
(12) Patent Application Publication
(10) Pub. No.: US 2012/0165559 A1
(43) Pub. Date: Jun. 28, 2012

(54) Compounds Having a Physiological Effect

(76) Inventors: Jean Mane, Grasse (FR); Jean-Claude Clinet, Villeneuve-Loubet (FR)

(21) Appl. No.: 13/265,636

(22) PCT Filed: Apr. 15, 2010
(86) PCT No.: PCT/FR10/00313

§ 371 Date: Jan. 12, 2012

(30) Foreign Application Priority Data
Apr. 23, 2009 (FR) 0952665

(51) Int. Cl. C07C 233/08; C07D 317/48; C07C 233/48

(52) U.S. Cl. 549/441; 564/123; 562/507; 560/125

(57) ABSTRACT

The present invention relates to compounds of general formula (I):

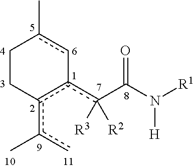

having a physiological effect, and to the preparation thereof and to the use of same.

The invention relates to compounds having particular physiological effects, causing in particular a cool or warm sensation on the skin and the mucous membranes, particularly of the mouth, of the nasal fossa and of the throat. The invention also relates to compositions containing these compounds and extends to various processes for synthesizing these compounds.

In the flavorings and fragrances industry, there is an increasing demand for products that give the user a pleasant effect of freshness. In the case of foods, encompassing drinks, confectionery, chewing gums, and also products intended for oral or body hygiene, substances capable of causing a cool sensation are firstly sought.

In this field, the reference substance is (L)-menthol, a predominant constituent of mint essential oils.

However, this compound has several drawbacks, such as a high volatility, a characteristic odor reminiscent of mint, and also a bitterness that develops at high concentration, making it unsuitable in formulations where these attributes are not desired. Moreover, a burning sensation is sometimes felt when (L)-menthol is used at a high concentration. In order to overcome these drawbacks, the flavorings and fragrances industry has studied the preparation of products which provide an intense and long-lasting physiological sensation of freshness but which have no bitterness or odor. Thus, certain derivatives of (L)-menthol have previously been reported, such as ethers [(L)-menthoxy-1,2-propanediol, (L)-menthoxy-2-methyl-1,2-propanediol, (L)-menthyl methyl ether], esters [(L)-menthyl acetate, lactate, succinate, glutarate, isobutyrate or 2-methoxyacetate], carbonates [(L)-menthyl glycerol carbonate], (L)-menthone and some of its acetals (lactic and glyceric), isopulegol and its acetate and also certain N-monosubstituted amides, such as 2,3-dimethyl-2-(2-propyl)butyric acid N-methylamide.

U.S. Pat. No. 4,150,052 describes N-mono-substituted amides of p-menthane-3-carboxylic acid as having a refreshing physiological effect. Among the many amides claimed by the company Wilkinson Sword Ltd., N-ethyl-p-menthane-3-carboxamide, called WS-3, is still very widely used to this day.

International application WO 2007/089252 lists a large number of compounds with a refreshing potential, and describes the use thereof for improving the taste of certain compositions such as medicaments.

Despite the existence of a significant number of refreshing physiological agents, there remains a need for compositions providing a cool sensation that lasts over time.

Moreover, there has for a few years been an increasing interest, on the world flavorings market, in very spicy preparations, causing a sensation of heat in the oral cavity. The compounds most widely used to this effect are capsaicin extracted from capsicums and piperine originating from pepper, but there is an established need for new strong taste sensations.

The invention falls within this search for novel compounds having particular physiological effects, in particular refreshing, warming and/or piquant effects. In this regard, the invention proposes compounds of general formula (I) hereinafter:

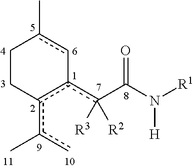

in which:
each of the dashed bonds is independently present or absent, provided that two successive dashed bonds are not simultaneously present;
R¹, which is other than an ethyl, is chosen from a C₁-C₆ alkyl, a C₂-C₆ alkenyl, a C₃-C₆ cycloalkyl and a C₅-C₆ cycloalkenyl; it being possible for each of these groups to be optionally substituted with at least one hydroxyl function, carboxyl function, nitrile function, C₁-C₄ alkoxy function, C₁-C₄ alkoxycarbonyl function, C₁-C₄ cyanoalkyl function and benzo[1,3]dioxol-5-yl function,
   if the dashed bond between the atoms C1 and C7 is absent,
R² and R³ are independently a hydrogen atom, or a C₁-C₆ alkyl,
   if the dashed bond between the atoms C1 and C7 is present,
R² is a hydrogen atom, or a linear or branched C₁-C₆ alkyl; and
R₃ is absent.

For the purpose of the present invention:
the term “C₁-C₆ alkyl” denotes any linear or branched alkyl group containing from 1 to 6 carbon atoms, in particular methyl, ethyl, n-propyl, isopropyl and isobutyl groups;
the term “C₂-C₆ alkenyl” denotes any linear or branched alkenyl group containing from 2 to 6 carbon atoms, comprising at least one alkenyl unsaturation, in particular ethylenyl, n-propylenyl, isopropylenyl and isobutylenyl groups;
the term “C₃-C₆ cycloalkyl” denotes any cyclic alkyl group containing from 3 to 6 carbon atoms, in particular cyclopentyl and cyclohexyl groups;
the term “C₅-C₆ cycloalkenyl” denotes any cyclic alkenyl group containing 5 or 6 carbon atoms, comprising at least one alkenyl unsaturation, in particular the phenyl group.

Similarly, the term “C₁-C₄ alkoxy”, “C₁-C₄ alkoxycarbonyl” or “C₁-C₄ cyanoalkyl” is intended to mean any alkoxy, alkyloxycarbonyl or cyanoalkyl group containing from 1 to 4 carbon atoms.

The present invention relates in particular to the compounds of general formulae (I-1), (I-2), (I-3), (I-4) and (I-5):

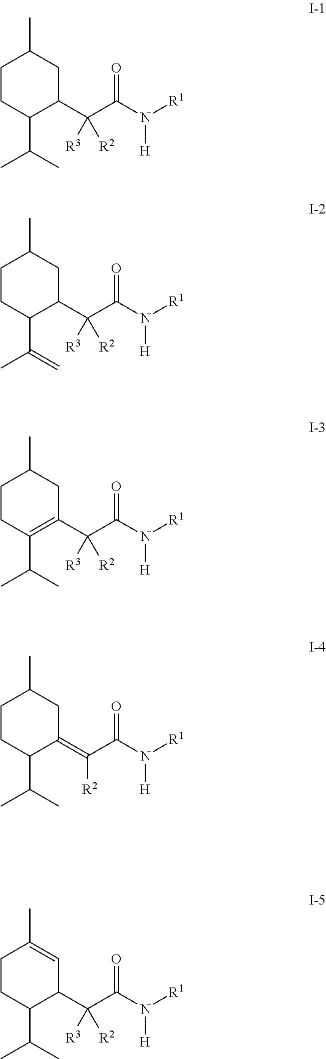

R¹, R² and R³ being as defined above.

Tables I, II, III, IV and V hereinafter list particularly advantageous examples of compounds according to the invention.

TABLE 1

| | |
|---|---|
| | I-1 |
| 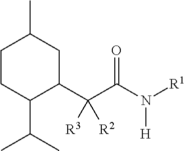 | |

| Compound | R¹ | R² | R³ | | Organoleptic qualities |
|---|---|---|---|---|---|
| 1 | —CH₃ | —H | —H | N-methyl-2-(2-isopropyl-5-methyl- | Intense coolness with |
| | | | | cyclohexyl)acetamide | delayed effect, |
| | | | | | persistent, note of mint |
| | | | | | and liquorice |
| 2 | —CH₂CH(CH₃)₂ | —H | —H | N-isobutyl-2-(2-isopropyl-5- | Refreshing and tingling |
| | | | | methylcyclohexyl)acetamide | |
| 3 | —CH₂COOH | —H | —H | [2-(2-isopropyl-5- | Warm |
| | | | | methylcyclohexyl)acetyl- | |
| | | | | amino]acetatic acid | |
| 4 | —CH₂COOCH₃ | —H | —H | [2-(2-isopropyl-5-methylcyclo- | Warm and piquant at the |
| | | | | hexyl)acetylamino]methyl acetate | back of the mouth, subtle |
| | | | | | freshness |
| 5 | —CH₂COOC₂H₅ | —H | —H | [2-(2-isopropyl-5-methylcyclo- | Persistent mentholated |
| | | | | hexyl)acetylamino]ethyl acetate | piquant, moderately cool |
| 6 | —CH₂CH₂OH | —H | —H | N-(2-hydroxyethyl)-2-(2-isopropyl- | Refreshing and tingling, |
| | | | | 5-methylcyclohexyl)acetamide | with an aniseed, |
| | | | | | liquorice note |
| 7 | —CH₂C(CH₃)₂OH | —H | —H | N-(2-hydroxy-2-methylpropyl)-2-(2- | Woody, piquant, and cold, |
| | | | | isopropyl-5-methylcyclohexyl)- | accompanied by a tingling |
| | | | | acetamide | stimulating salivation |
| 8 | —CH₃ | —CH₃ | —H | 2-(2-isopropyl-5-methylcyclo- | Piquant, slight |
| | | | | hexyl)-N-methylpropionamide | salivating effect |
| 9 | —CH₃ | —C₂H₃ | —H | 3-(2-isopropyl-5-methylcyclo- | Cool |
| | | | | hexyl) -N-methylbutyramide | |
| 10 | 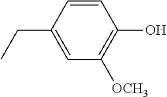 | —H | —H | N-(4-hydroxy-3-methoxybenzyl)-2- (2-isopropyl-5-methylcyclohexyl)- acetamide | Warm and piquant like pepper, with a salivating effect |
| 11 | 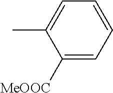 | —H | —H | N-(2-methyloxycarbonylphenyl)-2- (2-isopropyl-5-methylcyclohexyl)- acetamide | Warm |
| 12 | 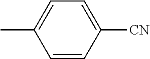 | —H | —H | N-(4-cyanophenyl)-2-(2-isopropyl- 5-methylcyclohexyl)acetamide | Warm |
| 13 | 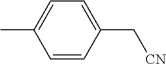 | —H | —H | N-(4-cyanomethylphenyl)-2-(2- isopropyl-5-methylcyclohexyl)- acetamide | Warm |
| 14 | 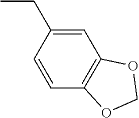 | —H | —H | N-benzo[1,3]dioxol-5-ylmethyl-2- (2-isopropyl-5-methylcyclohexyl)- acetamide | Warm and cold at the back of the throat, with a vanilla note |

TABLE II

| | |
|---|---|
| | I-2 |
| 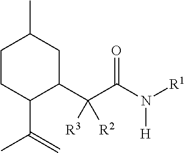 | |

| Compound | R¹ | R² | R³ | | Organoleptic qualities |
|---|---|---|---|---|---|
| 15 | —CH₃ | —H | —H | 2-(2-isopropenyl-5-methylcyclo- | Refreshing |
| | | | | hexyl)-N-methylacetamide | |
| 16 | 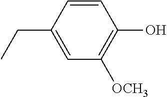 | —H | —H | N-(4-hydroxy-3-methoxybenzyl)-2- (2-isopropenyl-5-methylcyclo- hexyl) acetamide | Powerful piquant with delayed effect, salivating, slightly woody |

TABLE III

| | |
|---|---|
| 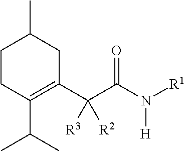 | I-3 |

| Com- | | | | | Organoleptic |
|---|---|---|---|---|---|
| pound | R¹ | R² | R³ | | qualities |
| 17 | —CH₃ | —H | —H | 2-(2-isopropyl-5- | Refreshing |
| | | | | methylcyclohex-1-enyl)- | |
| | | | | N-methylacetamide | |

TABLE IV

| | |
|---|---|
| 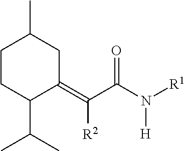 | I-4 |

| Com- | | | | Organoleptic |
|---|---|---|---|---|
| pound | R¹ | R² | | qualities |
| 18 | —CH₃ | —H | 2-(2-isopropyl-5-methylcyclo- | Refreshing |
| | | | hexylidene)-N-methylacetamide | |

TABLE V

| | |
|---|---|
| 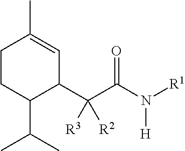 | I-5 |

| Compound | R¹ | R² | R³ | | Organoleptic qualities |
|---|---|---|---|---|---|
| 19 | —CH₃ | —H | —H | 2-(6-isopropyl-3-methylcyclohex-2- | Refreshing with a delayed |
| | | | | enyl)-N-methylacetamide | effect, slightly woody |

The invention is based on the following observation, made by the inventors and backed up by flavor experts, that the physiological effects of N-ethyl-2-(2-isopropyl-5-methylcyclohexyl)acetamide:

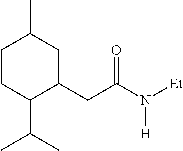

already known as a refreshing agent (and described in particular in international application WO 2007/089252 mentioned above), can be significantly and advantageously modulated by virtue of a chemical modification carried out precisely at the level of the ethyl group of the amide function.

The inventors have in particular noted that replacing this ethyl group with a group of smaller steric hindrance, namely a methyl group, results in a compound with notably amplified refreshing effects.

Conversely, replacing this ethyl group with a group of larger steric hindrance confers on the compound a particularly advantageous warming, spicy and/or tingling effect. In certain cases, a more or less pronounced refreshing effect is retained.

A first aspect of the invention therefore relates to compounds of general formula (I), as defined above, for which a high refreshing capacity has been noted. The R¹ group of these compounds is a methyl.

According to this first aspect of the invention, at least one of the R² and R³ groups is advantageously a hydrogen atom. In particular, R³ is a hydrogen atom and R² is chosen from a hydrogen atom and a methyl or ethyl group. Preferably, R² and R³ are two hydrogen atoms.

According to this first aspect of the invention, the preferred aspects are in particular:
N-methyl-2-(2-isopropyl-5-methylcyclohexyl)-acetamide (compound 1)
2-(6-isopropyl-3-methylcyclohex-2-enyl)-N-methylacetamide (compound 19)
2-(2-isopropenyl-5-methylcyclohexyl)-N-methylacetamide (compound 15)
2-(2-isopropyl-5-methylcyclohex-1-enyl)-N-methylacetamide (compound 17)
2-(2-isopropyl-5-methylcyclohexylidene)-N-methylacetamide (compound 18)
2-(2-isopropyl-5-methylcyclohexyl)-N-methylpropionamide (compound 8)
3-(2-isopropyl-5-methylcyclohexyl)-N-methylbutyramide (compound 9).

A comparative study shows that, among these refreshing agents, compound 1 is the most effective, both in terms of intensity and in terms of perception threshold.

A second aspect of the invention relates to compounds of general formula (I), as defined above, for which a warming and/or piquant and/or salivating effect has been noted. The R¹ group of these compounds is characterized by a steric hindrance that is greater than that of an ethyl radical. In particular, R¹ can be chosen from:
an isopropyl, isobutyl, ethylenyl, isopropenyl, isobutenyl, phenyl or benzyl group; it being possible for each of these groups to be optionally substituted with at least one group chosen from a hydroxyl, carboxyl, nitrile, C₁-C₄ alkoxy (in particular methoxy and ethoxy), C₁-C₄ alkoxycarbonyl (in particular methoxycarbonyl and ethoxycarbonyl) or C₁-C₄ cyanoalkyl function, or else
a methyl substituted with a group chosen from a hydroxyl, carboxyl, nitrile, C₁-C₄ alkoxy (in particular methoxy and ethoxy), C₁-C₄ alkoxycarbonyl (in particular methoxycarbonyl and ethoxycarbonyl) or C₁-C₄ cyanoalkyl function.

According to one preferred embodiment, R¹ is a phenyl or benzyl group substituted with at least one group chosen from a hydroxyl, carboxyl, nitrile, C₁-C₄ alkoxy (in particular methoxy and ethoxy), C₁-C₄ alkoxycarbonyl (in particular methoxycarbonyl and ethoxycarbonyl) and C₁-C₄ cyanoalkyl function.

Preferably, R¹ is chosen from

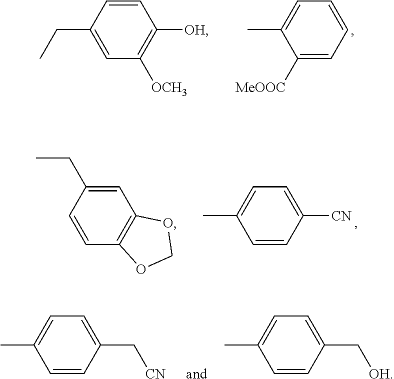

Generally, the compounds belonging to this embodiment are characterized by a rather warming and/or spicy physiological effect.

According to another preferred embodiment, R¹ is chosen from an isopropyl, isobutyl, —CH₂COOH, —CH₂COOMe, —CH₂COOEt, —CH₂OH, —CH₂CH₂OH and —CH₂C(CH₃)₂OH group. Generally, the compounds belonging to this embodiment are characterized by a rather tingling, salivating physiological effect.

Advantageously and according to the invention, R² and R³ are two hydrogen atoms.

According to this second aspect of the invention, the preferred compounds are in particular:
N-(4-hydroxy-3-methoxybenzyl)-2-(2-isopropyl-5-methylcyclohexyl)acetamide (compound 19)
N-(4-hydroxy-3-methoxybenzyl)-2-(2-isopropenyl-5-methylcyclohexyl)acetamide (compound 16)
N-benzo[1,3]dioxol-5-ylmethyl-2-(2-isopropyl-5-methylcyclohexyl)acetamide (compound 14)
[2-(2-isopropyl-5-methylcyclohexyl)acetylamino]-ethyl acetate (compound 5)
[2-(2-isopropyl-5-methylcyclohexyl)acetylamino]-methyl acetate (compound 4)
N-(2-hydroxy-2-methylpropyl)-2-(2-isopropyl-5-methylcyclohexyl)acetamide (compound 7)
N-(2-hydroxyethyl)-2-(2-isopropyl-5-methylcyclohexyl)acetamide (compound 6)
N-(4-cyanomethylphenyl)-2-(2-isopropyl-5-methylcyclohexyl)acetamide (compound 13)
N-(4-cyanophenyl)-2-(2-isopropyl-5-methylcyclohexyl)acetamide (compound 12)
[2-(2-isopropyl-5-methylcyclohexyl)acetylamino]acetatic acid (compound 3)
N-isobutyl-2-(2-isopropyl-5-methylcyclohexyl)-acetamide (compound 2)
N-(2-methyloxycarbonylphenyl)-2-(2-isopropyl-5-methylcyclohexyl)acetamide (compound 11).

Among these compounds, compounds 10 and 11 are particularly preferred. Compound 10 shows a strong piquant-burning physiological effect. Compound 11, for its part, exhibits a notable warming physiological effect, devoid of the piquant-burning aspect.

The compounds according to the invention have several asymmetric centers. The invention covers the various enantiomers and diastereoisomers, considered separately or as a mixture.

The invention extends to mixtures of enantiomers of general formula (I) as defined above, it being possible for each enantiomer to be present in said mixtures in varying proportions. A subject of the invention is in particular racemic mixtures of compounds of general formula (I) as defined above.

The production of the mixtures of enantiomers or pure forms is carried out by methods known to those skilled in the art using, for example, optically enriched or optically pure starting products, and methods of separation by crystallization or chromatography.

As mentioned above, the compounds of general formula (I) according to the invention have particular physiological effects. They cause a cool and/or warm sensation on the skin and the mucous membranes, particularly of the mouth, of the nasal fossa and of the throat. Some of these amides cause a more or less intense refreshing physiological effect giving the foods to which they are added a sensation of freshness, whereas others cause a warming physiological effect. Some of them can also cause other physiological effects, such as tingling or a piquant-burning physiological effect characteristic of substances that activate the trigeminal nerve, therefore giving the foods to which they are added a spicy sensation.

Consequently, the compounds according to the invention are advantageously used as physiological agents, in particular as refreshing agents or as warming agents, for example in compositions of food product type (chewing gums, confectionery, drinks, etc.), cosmetic, medicinal or paramedical compositions (bodycare or oro-dental hygiene compositions, lozenges, mouth sprays, syrups, lotions, etc.). The compounds according to the invention can be used alone or as a mixture with one or more other physiological agents (for example, flavorings, odorous agents) known to those skilled in the art, and that they are able to choose according to the desired effect.

The base product of such compositions will be readily determined by those skilled in the art according to the composition envisioned and therefore to the use envisioned, for which the usual components, such as solvent(s) and/or adjuvant(s), are well known. Suitable base products comprise, for example, and in a nonlimiting manner, food, medicinal, cosmetic, bodycare and oro-dental hygiene products.

The effective amount of the compounds according to the invention incorporated into the composition will vary according to the nature of the composition, the desired effect and the nature of the other compounds optionally present, and may be readily determined by those skilled in the art. Generally, this effective amount can vary within a very wide range, from 0.1 to 99% by total weight of the composition, in particular 0.1 to 50% by weight, especially 0.1 to 30% by weight.

The compounds according to the invention can be used as they are or else be incorporated into or onto an inert support material which can contain other ingredients suitable for the final composition. A large variety of support materials can be used, including, for example, polar solvents, oils, fats, finely divided solids, cyclodextrins, maltodextrins, gums, resins and any other support material known for such compositions.

The invention relates in particular to a cosmetic composition for caring for the face, the body or a part of the body, in particular a face and/or body cream, talcum powder, hair or body oil, shampoo, hair lotion, shower gel, toiletry soap, body antiperspirant, body deodorant, lotions, shaving cream, shaving soap, toothpaste, mouthwash or ointment, comprising at least one compound of formula (I) according to the invention, or a composition comprising at least one compound of general formula (I) according to the invention.

The invention also relates in particular to aromatic compositions of the food product type, or which are part of the composition of final food products (such as chewing gums, confectionery, alcoholic or nonalcoholic drinks, etc.), comprising at least one compound of general formula (I) according to the invention.

The invention also relates in particular to aromatic compounds which are part of the composition of medicinal or paramedical products, suitable for intraoral application (in particular syrup, tablet, lozenge, spray, mouthwash, toothpaste), which comprise at least one compound of general formula (I) according to the invention. The invention extends to the corresponding medicinal or paramedical products.

The invention extends to the methods for preparing the compounds of general formula (I) according to the invention.

In a first embodiment, the compounds of formula (I-1), with R² and R³ each denoting a hydrogen atom and R¹ being as defined above, can be obtained from the amide II by a successive reaction with a strong base and an alkylating agent according to scheme 1.

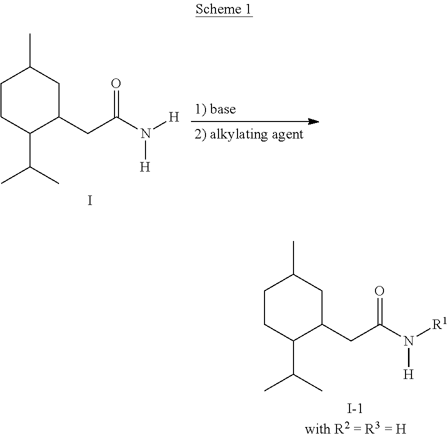

Preferentially, the base can be chosen from an amide, an alkali metal alkoxide or hydroxide, an organolithium compound or an alkali metal hydride. More preferentially, the base is chosen from organolithium compounds. Even more preferentially, the base is N-butyllithium. The alkylating agent may be any alkylating agent well known to those skilled in the art which makes it possible to alkylate the amine function with an alkyl R¹ as defined in formula (I). Preferably, the alkylating agent is chosen from a sulfonic ester (such as tosylate or mesylate), an alkyl bromide, an alkyl iodide or an alkyl chloride, provided that a catalytic amount of an alkali metal halide is simultaneously introduced into the reaction medium. More preferentially, the alkylating agent is an alkyl bromide or an alkyl iodide. A suitable solvent for this reaction may be an aromatic hydrocarbon, an ether, a cyclic ether, an aprotic polar solvent such as dimethylformamide or dimethyl sulfoxide, or a mixture thereof.

Generally, the compounds of formula (I-1) as obtained at the end of scheme 1 (and where R² and R³ are each a hydrogen atom, R¹ being as defined above) can be alkylated once or optionally twice so as to give the compounds of formula (I-1) according to scheme 2.

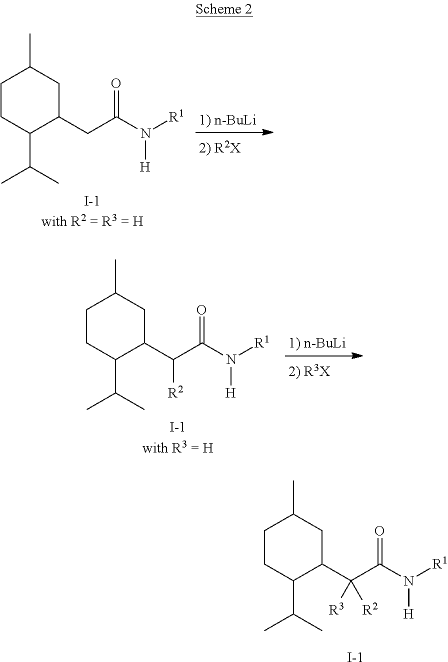

The alkylating agents making it possible to obtain the substitutions R² and R³ are well known to those skilled in the art. Preferably, the alkylating agent is chosen from a sulfonic ester (such as tosylate or mesylate), an alkyl bromide, an alkyl iodide or an alkyl chloride provided that a catalytic amount of an alkali metal halide is simultaneously introduced into the reaction medium. Preferentially, the alkylating agent is an alkyl bromide or an alkyl iodide.

The literature reports the synthesis of the dimethyl ester of [1r]-2-(trans-2-isopropenyl-cis-5-methylcyclohexyl)malonic acid III-2 from citronellal (L. F. Tietze, T. Eicher, U. Diederichsen and A. Speicher, Reactions and Synthesis in the Organic Chemistry Laboratory, p. 459, Wiley-VCH ed. 2007), an adaptation of which (E. J. Corey and P. Carpino, Tet. Lett., 31, 3857, 1990) describes a stereoselective synthesis route. Thus, depending on whether the starting product is (S)-citronellal, (R)-citronellal or a racemic mixture of citronellal, the compound III-2 is obtained, respectively, in enantiopure (1S,2S,5S) or (1R,2R,3R) form or in the form of a racemic mixture thereof. Hydrogenation of the compound III-2 subsequently produces the dimethyl ester of [1r]-2-(trans-2-isopropyl-cis-5-methylcyclohexyl)malonic acid III-1 (scheme 3).

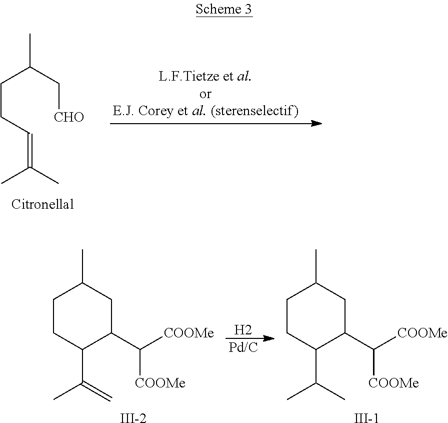

In a second embodiment, the compounds of formulae (I-1) and (1-2) can be obtained from the malonic esters of formulae (III-1) and (III-2) respectively. After a series of reactions well known to those skilled in the art which make it possible to obtain the acids of formula (IV), the amides of formulae (I-1) and (1-2) were produced by activation of the carboxyl function of the compounds of formula (IV), followed by a condensation with the appropriate amine according to scheme 4.

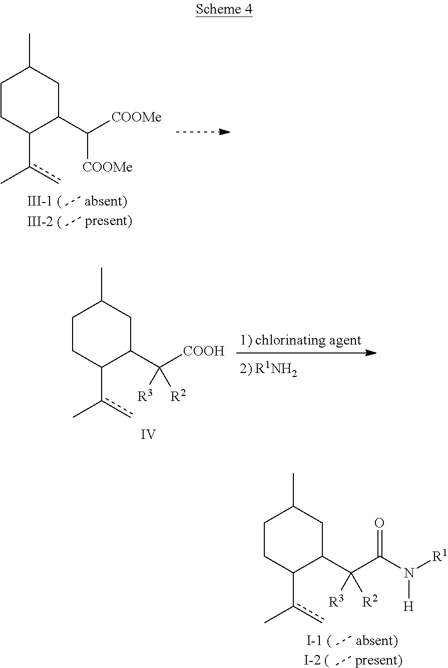

The chlorinating agent may be any chlorinating agent well known to those skilled in the art, such as SOCl₂, ClCO—COCl, PCl₃ or PCl₅; more preferentially, the chlorinating agent is SOCl₂ or ClCO—COCl.

A third embodiment makes it possible to obtain the compounds of formula (I) via an intermediate amide alcohol of formula (VI) obtained by condensation of an alkylamide of formula

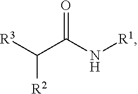

in which R¹, R² and R³ are as defined in formula (I), with a ketone of formula (V), in which each of the dashed bonds is independently present or absent with the proviso that two successive pointed bonds are not simultaneously present. The compounds of formula (V) that are preferred are the compounds (Va) to (Vf) defined in table VI.

TABLE VI

| | Compound | | | | | |
|---|---|---|---|---|---|---|
| | Va | Vb | Vc | Vd | Ve | Vf |
| Formula | 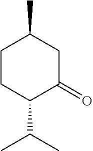 | 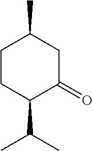 | 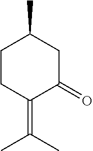 | 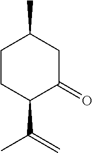 | 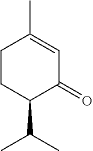 | 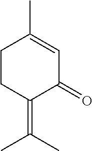 |

The intermediate amide alcohol can subsequently be subjected to various steps that a person skilled in the art will be able to choose in order to obtain the desired compounds of general formula (I) (scheme 5).

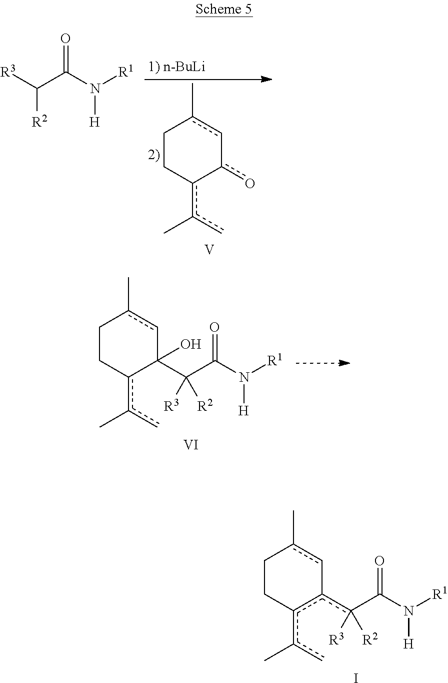

In particular, the amide alcohol may be, for example, subjected to a dehydration in an acidic medium, or to a successive reaction with carbonyldiimidazole and potassium t-butoxide (with the proviso that R³ is a hydrogen atom); it being possible for these steps to be optionally followed by a more or less controlled hydrogenation in order to obtain other compounds of general formula (I), in particular the compounds of formula (I-1).

In a first variant of this embodiment, the alkylamide of formula

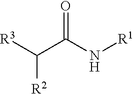

is condensed with a ketone of formula (Va) and the amide alcohol thus obtained is subjected to a dehydration in an acidic medium, resulting in the compound of formula (I-3):

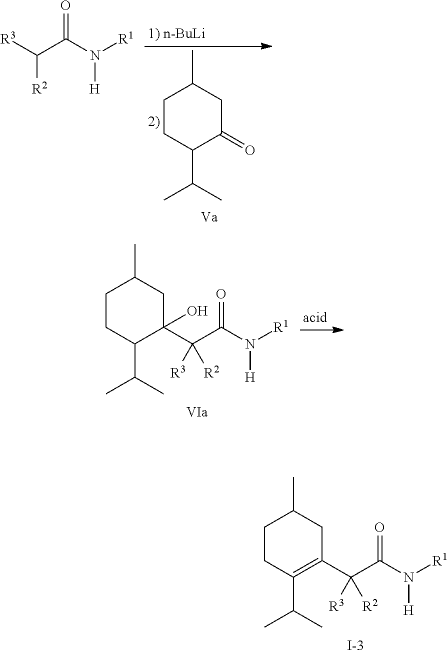

In a second variant of this embodiment, the alkylamide of formula

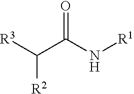

is condensed with a ketone of formula (Va) and the amide alcohol thus obtained is subjected to a successive reaction with carbonyldiimidazole and potassium t-butoxide (with the proviso that R³ is a hydrogen atom), resulting in the compound of formula (I-4):

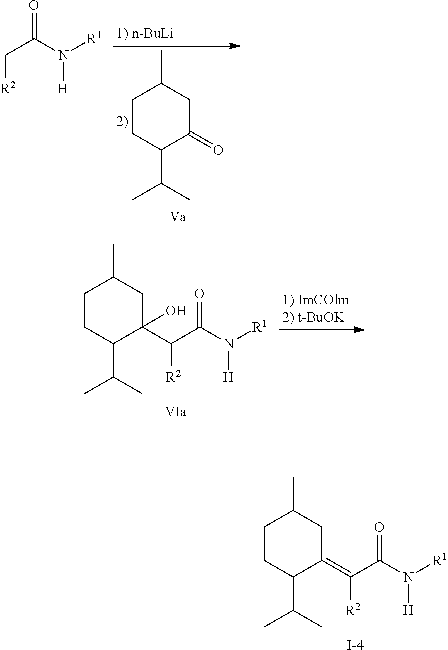

It is a fact acknowledged by those skilled in the art that enantiomers can have quite different organoleptic and physiological properties. Thus, while (L)-menthol is a physiologically refreshing agent, its enantiomer (D)-menthol is bitter and is barely used in aromatic compositions.

Since the compounds of formulae (I-1) and (1-2) comprise at least three asymmetric centers on the carbons C1, C2 and C5, the (1S,2S,5S) and (1R,2R,5R) enantiomers can be obtained selectively from, respectively, (S)-citronellal and (R)-citronellal according to schemes 3 and 4. The racemic mixture of the (1S,2S,5S) and (1R,2R,5R) enantiomers can for its part be obtained in the same way from racemic citronellal. The enantiomers can then be separated according to methods known to those skilled in the art, such as crystallization or chromatography methods. Generally, the (1S,2S,5S) and (1R,2R,5R) enantiomers of the same racemic mixture as the latter do not exhibit any notable difference with regard to the organoleptic and physiological qualities, both in terms of perception threshold and of intensity of the refreshing effect felt. Consequently, a subject of the present invention is any pure enantiomer and diastereoisomer of the compounds of formula (I) and also any mixture thereof in any proportion, in particular racemic mixtures.

The following examples further illustrate the various processes for producing the compounds according to the invention and also the use thereof, and the advantage thereof. These examples are given only for the purpose of illustration and cannot be considered to limit the invention.

### EXAMPLE 1

### Preparation of Compound II

Firstly, compound III-2 is obtained from racemic citronellal according to scheme 3. Compound II is then obtained according to scheme 4. The reaction scheme detailed is the following:

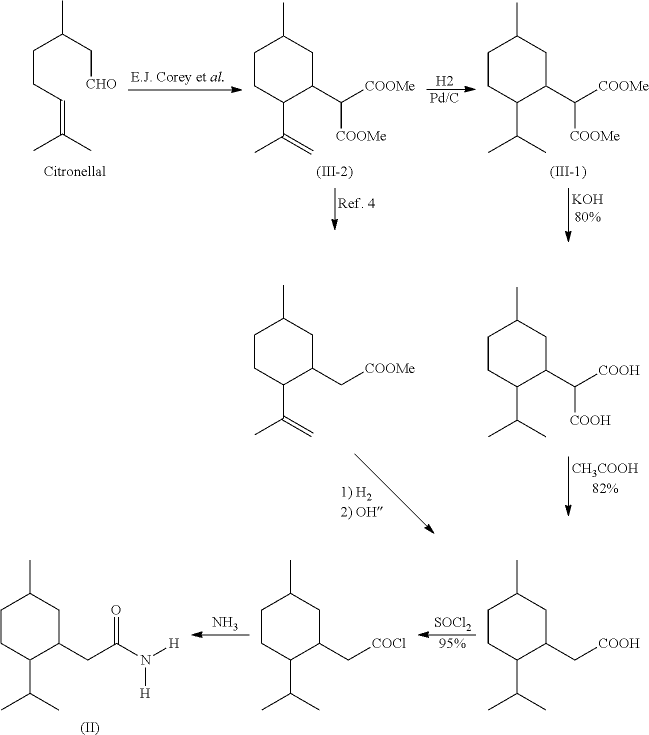

### Preparation of [1r]-2-(trans-2-isopropyl-cis-5-methylcyclohexyl)acetic acid

Method 1

7 g of palladium at 5% on carbon, 134 g (0.5 mol) of compound III-2 and 0.5 l of methanol are successively introduced into a 1 l autoclave. The autoclave is conditioned under 3 bar of hydrogen and stirred for 24 hours. The solution is filtered and concentrated and the residual product is distilled under vacuum. 128 g of compound III-1 are isolated, i.e. a molar yield of 95%.

63 g of KOH (1.12 mol, 1.5 eq.), 63 g of water, 0.74 l of methanol and then 100 g (0.37 mol) of compound III-1 are successively introduced into a conventional apparatus. The mixture is brought to reflux for 12 hours, and then the condenser is replaced with a distillation apparatus. The methanol is distilled off, replacing it with water as the distillation proceeds. The distillation is interrupted when the temperature of the vapors reaches 80° C. The solution, cooled to ambient temperature, is extracted twice with 100 ml of methyl t-butyl ether, and then cooled to 0° C. and acidified to pH=3. The suspension obtained is extracted three times with 200 ml of methyl t-butyl ether. The organic phase is washed to neutrality, dried over magnesium sulfate, and then concentrated. The residual solid is recrystallized from a mixture of methyl t-butyl ether and methylcyclohexane. 71.5 g of [1r]-2-(trans-2-isopropyl-cis-5-methylcyclohexyl)malonic acid are obtained in the form of a white solid, i.e. a yield of 80%.

120 g of acetic acid and 3 g of 96% sulfuric acid (0.031 mol) are successively introduced into a conventional apparatus. The solution is stirred for 5 minutes and then 80 g (0.33 mol) of [1r]-2-(trans-2-isopropyl-cis-5-methylcyclohexyl)malonic acid previously obtained are added. The mixture obtained is brought to reflux for 12 hours and is then cooled to ambient temperature, and then 5 g of sodium acetate trihydrate are added. The resulting solution is stirred for 0.5 hour. The acetic acid is distilled off under vacuum and the residue is taken up into 0.2 l of water which is subsequently extracted with three times 0.15 l of methyl t-butyl ether. After drying over magnesium sulfate, the solution is concentrated and the residue is then distilled under vacuum. The viscous oil obtained is crystallized from methylcyclohexane at −30° C. so as to produce 53.7 g of [1r]-2-(trans-2-isopropyl-cis-5-methylcyclohexyl)acetic acid in the form of a white solid, thereby representing a yield of 82%.

Method 2

25 g of compound III-2, 3 g of Pd/C at 5% and then 0.1 l of anhydrous toluene are introduced into a 0.25 l autoclave. The autoclave is conditioned under 3 bar of hydrogen and then stirred for 24 hours. After verification of the absence of starting product, the mixture is filtered, concentrated and distilled. 23.4 g of the methyl ester of [1r]-2-(trans-2-isopropenyl-cis-5-methylcyclohexyl)acetic acid are isolated in the form of a colorless oil, i.e. a yield of 92%.

Bp=56° C./26.6 Pa

5.3 g of KOH (2 eq.), 10 g of water, 0.1 l of methanol and then 10 g (0.047 mol) of the methyl ester of [1r]-2-(trans-2-isopropenyl-cis-5-methylcyclohexyl)acetic acid previously obtained are successively introduced into a conventional apparatus. The resulting solution is brought to reflux for 3 hours. The methanol is then removed by distillation after dilution with 0.1 l of water. The resulting aqueous phase is extracted with twice 0.05 l of methyl t-butyl ether and then acidified to pH=3. The white suspension is taken up with twice 0.1 l of methyl t-butyl ether. This organic phase is washed to neutrality, dried, and then concentrated. The white residue is recrystallized from methylcyclohexane so as to give 7.9 g of [1r]-2-(trans-2-isopropyl-cis-5-methylcyclohexyl)acetic acid.

Yield: 85%.

### Preparation of Compound II

40 g (0.202 mol) of [1r]-2-(trans-2-isopropyl-cis-5-methylcyclohexyl)acetic acid previously obtained, 0.2 l of anhydrous toluene and then 3 drops of dimethylformamide are introduced successively into a conventional apparatus. Freshly purified thionyl chloride (37.5 g, 1.3 eq.) is poured into the dropping funnel and then added dropwise to the round-bottomed flask. The resulting mixture is stirred for 12 hours at ambient temperature and then the solvent is distilled off under partial vacuum. The residue is distilled under high vacuum so as to give 42.5 g of [1r]-2-(trans-2-isopropyl-cis-5-methylcyclohexyl)acetyl chloride in the form of a colorless liquid, i.e. a yield of 98%.

0.05 l of a 32% solution of ammonia in water is introduced into a conventional apparatus, said solution being brought to 0° C. by means of a water/ice bath. A solution of 21 g (0.1 mol) of [1r]-2-(trans-2-isopropyl-cis-5-methylcyclohexyl)acetyl chloride, previously obtained, in 0.15 l of anhydrous dichloromethane is introduced into the dropping funnel and then added dropwise to the round-bottomed flask. The resulting mixture is stirred for 2 hours and the phases are then separated. The aqueous phase is retained. The organic phase is washed twice with 0.1 l of clear water, 0.1 l of 5% hydrochloric acid and then again with 0.1 l of clear water. This organic phase is dried over magnesium sulfate and then concentrated. The residual solid is recrystallized from a mixture of methyl t-butyl ether and methylcyclohexane so as to give 14.60 g of compound II.

Mp=147° C.; yield 74%.

¹H NMR: CDCl₃, 200 MHz: 0.77 (d; 7.0 Hz, 3H); 0.86 (d, 6.8 Hz, 3H); 0.91 (d, 6.8 Hz, 3H); from 0.60 to 1.10 (m, 4H); 1.20 to 1.50 (m, 1H); 1.60 to 2.00 (m, 6H); 2.55 (d*d, 8.4/18.5 Hz, 1H); 5.7 (s, 2H)

¹³C NMR: CDCl₃, 50 MHz: 15.75; 21.95; 22.96; 24.62; 27.30; 32.88; 35.59; 37.34; 40.91; 42.15; 47.61; 176.13 ppm.

MS: m/z=197 [M⁺]; 182; 165; 154; 138; 112; 95; 86; 67; 59 (100) amu.

IR: σ(cm⁻¹) 3400, 3200, 1647, 1438, 1410, 1194, 1152, 801, 693, 657.

### EXAMPLE 2

### Preparation of Compound 1

Method 1

According to a protocol identical to that of the preparation of compound II described in example 1, with the 0.05 l of aqueous ammonia being replaced with the same volume of a 40% aqueous solution of monomethylamine, 9.7 g of compound I are obtained in the form of a white solid after recrystallization using a mixture of methyl t-butyl ether and methylcyclohexane. Mp=142° C.; yield 92%.

¹H NMR: CDCl₃, 200 MHz: 0.75 (d, 6.8 Hz, 3H); 0.83 (d, 6.7 Hz, 3H); 0.88 (d, 6.7 Hz, 3H; from 0.5 to 1.2 (m, 3H); 1.45 (m, 1H); 1.50 to 2.00 (m, 7H), 2.50 (m, 1H); 2.79 (d, 4.8 Hz, 3H); 5.56 (s, 1H).

¹³C NMR: CDCl₃, 50 MHz: 15.27; 21.60; 22.60; 24.30; 26.30; 26.90; 32.52; 35.26; 37.07; 41.18; 41.81; 47.29; 173.81 ppm.

MS: m/z=211 [M⁺]; 196; 180; 168; 163; 126; 100; 95; 81; 73(100); 58 amu

IR: σ(cm⁻¹) 3254, 3090, 1643, 1568, 1454, 1372, 1279, 1189, 1160, 995, 913, 753, 722, 626.

Method 2

According to scheme 1, 3.94 g of (0.02 mol) of compound II obtained in example 1 and 0.05 l of anhydrous tetrahydrofuran are introduced successively into a conventional apparatus. The solution is brought to 0° C. and then 0.009 l of N-butyllithium in a 2.25 N solution in hexane (1.05 eq.) is slowly added. The mixture is stirred for one hour at the same temperature and then 3.55 g (1.25 eq.) of iodomethane are added rapidly. The resulting suspension is stirred for two hours at ambient temperature and is then hydrolyzed, dried and concentrated. The white solid obtained is recrystallized from a mixture of methyl t-butyl ether and methylcyclohexane so as to give 3.55 g of compound 1, i.e. a yield of 84%. The product is identical to the product obtained using the first method.

### EXAMPLE 3

### Preparation of Compound 2

According to a protocol identical to that of the preparation of compound II described in example 1, using 9.14 g (0.125 mol, 2.5 eq.) of isobutylamine (as a replacement for the aqueous ammonia), compound 2 is obtained.

Mp=82° C.; yield 83%.

¹H NMR: CDCl₃, 200 MHz: 0.75 (d, 6.7 Hz, 3H); 0.82 (d, 6.7 Hz, 3H); 0.90 (d, 2.8 Hz, 3H); 0.95 (d, 2.8 Hz, 3H), from 0.50 to 1.20 (m, 6H); from 1.20 to 1.50 (m, 2H); from 1.50 to 2.00 (m, 7H), 2.50 (d, 12.3 Hz, 1H); 3.10 (m, 2H); 5.6 (s, 1H).

¹³C NMR: CDCl₃, 50 MHz: 15.66; 20.52; 21.95; 22.97; 24.65; 27.29; 28.90; 32.89; 35.62; 37.44; 41.85; 42.37; 47.24; 47.71; 173.32 ppm.

MS: m/z=253 [M⁺]; 238; 224; 210; 198; 181; 168; 163; 142; 137; 115(100); 100; 95; 81; 72; 60 amu.

IR: σ(cm⁻¹) 3315, 3093, 2955, 2847, 1642, 1553, 1464, 1437, 1370, 1275, 1187, 1160, 1126, 997, 973, 738, 704, 627.

### EXAMPLE 4

### Preparation of Compound 3

According to a protocol identical to that of example 3, using 9.46 g (0.125 mol, 2.5 eq.) of glycine, compound 3 is obtained.

Mp=153° C.; yield 44%.

¹H NMR: CDCl₃, 200 MHz: 0.70 (d, 6.8 Hz, 3H); 0.81 (d, 6.6 Hz, 3H); 0.86 (d, 6.6 Hz, 3H); from 0.50 to 1.10 (m, 4H); from 1.10 to 1.30 (m, 1H); from 1.50 to 2.10 (m, 6H); 2.32 (m, 1H); 3.70 (m, 2H); 8.2 (m, 2H).

¹³C NMR: CDCl₃, 50 MHz: 15.50; 21.83; 22.97; 24.27; 26.58; 26.62; 32.50; 35.22; 36.69; 36.82; 46.95; 169.70; 172.69 ppm.

IR: σ(cm⁻¹) 3366, 3279, 3100, 2950, 2850, 1748, 1703, 1662, 1601, 1528, 1439, 1337, 1214, 1133, 1035, 995, 668, 610.

### EXAMPLE 5

### Preparation of Compound 4

According to a protocol identical to that of example 3, using 9.42 g of methyl glycinate chloride (0.075 mol, 1.5 eq.) and 12.65 g (0.125 mol, 2.5 eq.) of triethylamine, compound 4 is obtained.

Mp=88° C.; yield 76%.

¹H NMR: CDCl₃, 200 MHz: 0.76 (d, 7.0 Hz, 3H); 0.85 (d, 6.7 Hz, 3H); 0.90 (d, 6.7 Hz, 3H); from 0.60 to 1.10 (m, 3H); from 1.20 to 1.50 (m, 2H); from 1.50 to 2.00 (m, 6H); 2.56 (d*d, 19/8.9 Hz, 1H); 3.77 (s, 3H); 4.06 (d, 5 Hz, 2H); 5.94 (s, 1H).

¹³C NMR: CDCl₃, 50 MHz: 15.25; 21.57; 24.24; 26.93; 32.50; 35.20; 37.05; 40.88; 41.23; 41.74; 47.16; 52.36; 170.62; 173.19 ppm.

MS: m/z=269 [M⁺]; 254; 238; 226; 210; 180; 163; 158; 137; 131(100); 109; 103; 99; 95; 90; 81 amu.

IR: σ(cm⁻¹) 3325, 3266, 3080, 2950, 2845, 1744, 1635, 1531, 1437, 1386, 1227, 1183, 1136, 980, 743, 7087, 671, 624.

### EXAMPLE 6

### Preparation of Compound 5

According to a protocol identical to that of example 3, using 10.46 g (0.075 mol, 1.5 eq.) of ethyl glycinate chloride, compound 5 is obtained.

Mp=79° C.; yield 69%.

¹H NMR: CDCl₃, 200 MHz: 0.76 (d, 6.8 Hz, 3H); 0.84 (d, 6.8 Hz, 3H); 0.90 (d, 6.8 Hz, 3H); from 0.60 to 1.10 (m, 3H); from 1.20 to 1.50 (m, 2H); 1.30 (t, 7.2 Hz, 3H); 1.50 to 2.00 (m, 6H); 2.57 (d*d, 18.9/8.9 Hz, 1H); 4.04 (d, 5 Hz, 2H); 4.22 (q, 7.2 Hz, 2H); 5.99 (s, 1H).

¹³C NMR: CDCl₃, 50 MHz: 14.17; 15.25; 21.57; 22.59; 24.24; 26.92; 32.50; 35.21; 37.03; 40.86; 41.40; 41.74; 47.15; 61.50; 170.17; 173.17 ppm.

MS: m/z=283 [M⁺]; 268; 240; 238; 210; 181; 172; 163; 145(100); 137; 117; 109; 104; 99; 95; 81 amu.

IR: σ(cm⁻¹) 3337, 3068, 1731, 1634, 1544, 1441, 1370, 1351, 1309, 1249, 1186, 1152, 11312, 1044, 1027, 944, 859, 655, 624, 596.

### EXAMPLE 7

### Preparation of Compound 6

According to the protocol of example 3, using 7.64 g (0.125 mol, 2.5 eq.) of anhydrous ethanolamine, compound 6 is obtained.

Mp=106° C.; yield 88%.

¹H NMR: CDCl₃, 200 MHz: 0.76 (d, 6.8 Hz, 3H); 0.85 (d, 6.6 Hz, 3H); 0.90 (d, 6.6 Hz, 1H); from 0.60 to 1.10 (m, 3H); from 1.20 to 1.50 (m, 2H); from 1.50 to 2.00 (m, 6H); 2.53 (d*d, 18.4/8.6, 1H); 3.39 (m, 2H); 4.20 (m, 2H); 6.75 (s, 1H).

¹³C NMR: CDCl₃, 50 MHz: 15.59; 21.91; 22.99; 24.57; 27.24; 32.86; 35.53; 37.37; 41.45; 42.03; 42.81; 47.68; 62.33; 175.05 ppm.

MS: m/z=241 [M⁺]; 226; 210; 19+8; 181; 163; 156; 137; 130; 109; 103(100); 95; 81; 60 amu.

IR: σ(cm⁻¹) 3447, 3302, 3084, 2950, 2850, 1623, 1554, 1452, 1278, 1218, 1190, 1132, 1060, 999, 966, 865, 747, 695, 624.

### EXAMPLE 8

### Preparation of Compound 7

According to the protocol of example 3, using 11.12 g (0.075 mol, 1.5 eq.) of 1-amino-2-methylpropan-2-ol, compound 7 is obtained.

Mp=110° C.; yield 88%.

¹H NMR: CDCl₃, 200 MHz: 0.77 (d, 7 Hz, 3H); 0.84 (d, 6.7 Hz, 3H); 0.90 (d, 6.7 Hz, 3H); from 0.60 to 1.10 (m, 3H); from 1.20 to 1.50 (m, 2H); 1.22 (s, 6H); from 1.60 to 2.00 (m, 6H); 2.55 (d*d, 18.8/8.9 Hz, 1H); 3.21 (s, H); 3.26 (d, 5.8 Hz, 2H); 6.25 (s, H).

¹³C NMR: CDCl₃, 50 MHz: 15.67; 21.96; 24.61; 27.30; 27.68 (2c); 32.86; 35.59; 37.37; 41.65; 42.23; 47.70; 50.88; 71.19; 174.77 ppm.

MS: m/z=269 [M⁺]; 254; 250; 236; 226; 211(100); 196; 181; 168; 163; 137; 131; 126; 113; 100; 95; 81; 73 amu.

IR: σ(cm⁻¹) 3321, 3086, 2963, 2843, 1639, 1557, 1445, 1368, 1275, 1148, 1132, 996, 741, 693, 658.

### EXAMPLE 9

### Preparation of Compound 10

According to the protocol of example 3, starting from 15.3 g of vanillylamine and 15.3 g of triethylamine, compound 10 is obtained.

Mp=140° C.; yield 68%.

¹H NMR: CD₃SOCD₃, 200 MHz: 0.54 (d, 6.8 Hz, 3H); 0.64 (d, 6.8 Hz, 3H); 0.69 (d, 6.8 Hz, 3H); from 0.60 to 1.10 (m, 5H); from 1.10 to 1.20 (m, 1H); from 1.30 to 1.80 (m, 5H); 2.10 to 2.30 (m, 1H); 3.58 (s, 3H); 4.02 (m, 2H); from 6.45 to 6.65 (m, 3H); 8.05 (s, 1H); 8.69 (s, 1H).

¹³C NMR: CD₃SOCD₃, 50 MHz: 15.50; 21.82; 22.95; 24.26; 26.59; 32.51; 35.19; 36.87; 40.27; 41.70; 42.19; 47.08; 55.85; 112.01; 115.43; 120.13; 130.98; 145.71; 147.76; 172.08 ppm.

IR: σ(cm⁻¹) 3333, 3139, 2951, 2912, 2841, 1633, 1601, 15550, 1515, 1451, 1433, 1371, 1349, 1273, 1250, 1234, 1188, 1156, 1124, 1034, 919, 866, 830, 798, 742.

### EXAMPLE 10

### Preparation of Compound 11

According to the protocol of example 3, starting from 15.1 g of methyl anthranilate and 15.3 g of triethylamine, compound 11 is obtained.

Mp=83° C.; yield 88%.

¹H NMR: CDCl₃, 200 MHz: 0.84 (d, 6.4 Hz, 3H); 0.86 (d, 6.8 Hz, 3H); 0.97 (d, 6.8 Hz, 3H); from 0.60 to 1.10 (m, 5H); from 1.10 to 1.20 (m, 1H); from 1.50 to 2.00 (m, 5H); 2.78 (m, 1H); 3.92 (s, 3H); 7.06 (t*d, 7.0/1.0 Hz, 1H); 7.53 (t*d, 7.0/1.6 Hz, 1H); 8.02 (d*d, 8.0/1.6 Hz, 1H); 8.76 (d*d, 8.0/1.0 Hz, 1H); 11.1 (s, 1H).

IR: σ(cm⁻¹) 3270, 2955, 2908, 1703, 1687, 1602, 1588, 1524, 1446, 1257, 1236, 1086, 764, 728, 702.

### EXAMPLE 11

### Preparation of Compound 12

According to a protocol similar to that of example 3 and starting from 3.10 g of 4-aminobenzonitrile and 2.78 g of anhydrous triethylamine, compound 12 is obtained.

5.41 g (0.025 mol) of [1r]-2-(trans-2-isopropyl-cis-5-methylcyclohexyl)acetic acid chloride, 0.05 l of anhydrous dichloromethane and 2.78 g (0.00385 l) of anhydrous triethylamine are introduced successively into a 250 ml three-necked round-bottomed flask equipped with an immersed thermometer, a vertical condenser equipped with a drying guard filled with a solution of KOH and a 0.1 l isobaric dropping funnel. The resulting solution is brought to 0° C.

A solution of 3.1 g (0.0265 mol) of 4-aminobenzonitrile in 0.02 l of anhydrous dichloromethane is then introduced into the funnel. This solution is added dropwise to the round-bottomed reaction flask, over the course of 0.5 hour, causing the appearance of a white precipitate. The solution obtained is stirred for 2 hours at ambient temperature, and is then washed successively with 0.1 l of water, 0.1 l of 1N HCl and 0.1 l of a saturated solution of sodium hydrogen carbonate in water. The organic phase is dried over anhydrous magnesium sulfate and then concentrated on a rotary evaporator. The white precipitate obtained is crystallized using a dichloromethane/hexane mixture at −20° C. A white solid is then isolated.

Mp=141° C.; yield 84%.

¹H NMR: CDCl₃, 200 MHz: 0.77 (d, 8 Hz, 3H); 0.83 (d, 7 Hz, 3H); 0.91 (d, 8 Hz, 3H); from 0.65 to 1.10 (m, 3H); from 1.20 to 1.50 (m, 2H); from 1.50 to 2.00 (m, 6H); 2.72 (d*d, 18/20 Hz, 1H); 3.10 (m, 2H); 7.58 (d, 9 Hz, 2H); 7.72 (d, 9 Hz, 2H); 7.92 (s, 1H).

¹³C NMR: CDCl₃, 50 MHz: 15.23; 21.54; 24.19; 27.04; 32.49; 35.08; 37.12; 41.90; 42.26; 47.14; 106.67; 119.56; 133.23; 142.28; 172.10 ppm.

IR: σ(cm⁻¹) 3247, 3178, 3106, 2959, 2911, 2842, 2218, 1663, 1594, 1537, 1501, 1444, 1409, 1354, 1325, 1307, 1262, 1251, 1174, 1121, 980, 846, 836, 776.

### EXAMPLE 12

### Preparation of Compound 13

According to the protocol of example 11, starting from 3.50 g of 4-aminobenzylamide (as a replacement for the 4-aminobenzylnitrile), compound 13 is obtained.

Mp=142° C.; yield 78%.

¹H NMR CDCl₃, 200 MHz: 0.77 (d, 8 Hz, 3H); 0.83 (d, 8 Hz, 3H); 0.94 (d, 8 Hz, 3H); from 0.60 to 1.10 (m, 3H); from 1.20 to 1.50 (m, 2H); from 1.50 to 2.00 (m, 6H); 2.67 (d*d, 18/10 Hz, 1H); 3.71 (s, 2H); 7.23 (d, 8 Hz, 2H); 7.56 (d, 8 Hz, 2H); 7.79 (s, 1H).

¹³C NMR: CDCl₃, 50 MHz: 15.24; 21.56; 22.57; 23.07; 24.20; 26.98; 32.48; 35.14; 37.14; 41.84; 42.12; 47.17; 118.02; 120.45; 125.19; 128.48; 138.02; 171.88 ppm.

IR: σ(cm⁻¹) 3344, 2958, 2912, 2840, 2250, 1660, 1597, 1520, 1413, 1370, 1322, 1306, 1173, 1126, 909, 809, 678.

### EXAMPLE 13

### Preparation of Compound 14

According to a protocol similar to that of example 3 and starting from piperonylamine, anhydrous dichloromethane and N-methylmorpholine, compound 14 is obtained.

5 g (0.0331 mol) of piperonylamine, 100 ml of anhydrous dichloromethane and 3.36 g (0.0331 mol) of N-methylmorpholine are introduced successively into a 250 ml three-necked round-bottomed flask which is equipped conventionally, dry and flushed with nitrogen. The mixture is brought to 0° C. 0.0331 mol of bromomentholic acid chloride, in solution in approximately 2 volumes of anhydrous dichloromethane, is added dropwise. A white precipitate forms immediately, and the addition is continued without exceeding 10° C. in the reaction medium. The solution obtained is stirred overnight at ambient temperature, and is then poured over a mixture of crushed ice and 10% aqueous HCl. The organic phase is then washed with 100 ml of water (which are added at the 1^{st} wash), 100 ml of 4N aqueous sodium hydroxide and 100 ml of water (added at the 3^{rd} wash), and then dried over MgSO₄ and concentrated on a rotary evaporator. The resulting white solid is recrystallized from a dichloromethane/methylcyclohexane mixture.

Mp=133° C.; yield 87%.

¹H NMR: CDCl₃, 200 MHz: 0.76 (d, 8 Hz, 3H); 0.84 (d, 8 Hz, 3H); 0.89 (d, 8 Hz, 3H); from 0.60 to 1.10 (m, 3H); from 1.20 to 1.50 (m, 2H); from 1.50 to 2.00 (m, 6H); 2.55 (m, 1H); 4.34 (d, 6 Hz, 2H); 5.85 (s, 1H); 5.93 (s, 2H); 6.76 (m, 3H).

¹³C NMR: CDCl₃, 50 MHz: 15.26; 21.55; 22.57; 24.20; 26.89; 32.47; 37.17; 37.06; 41.29; 41.82; 43.35; 47.28; 101.03; 108.23; 108.44; 121.07; 132.48; 146.89; 147.88; 172.83 ppm.

IR: σ(cm⁻¹) 3313, 2955, 2939, 2907, 2841, 1637, 1542, 1501, 1486, 1441, 1250, 1188, 1096, 1042, 942, 922, 856, 812, 734, 703.

### EXAMPLE 14

### Preparation of Compound 16

According to a protocol similar to that of examples 11 to 13, compound 16 is prepared.

6.3 g (0.075 mol) of sodium hydrogen carbonate, 0.1 l of distilled water, 5 g (0.02625 mol) of 4-hydroxy-3-methoxybenzylammonium chloride and 0.05 l of tetrahydrofuran are introduced successively into a 250 ml three-necked round-bottomed flask equipped with an immersed thermometer and an ascending condenser. The whole is stirred until a clear solution is obtained, and is then brought to 0° C. A solution of [1r]-2-(trans-2-isopropenyl-cis-5-methylcyclohexyl)acetyl chloride (5.36 g; 0.025 mol) in 0.05 l of tetrahydrofuran is added dropwise to the round-bottomed reaction flask.

The mixture is stirred for 2 hours at ambient temperature, and then the solvent is eliminated on a rotary evaporator. The resulting aqueous phase is extracted with twice 0.1 l of dichloromethane. The combined organic phases are washed with 0.1 l of 1N hydrochloric acid and then 0.1 l of a saturated aqueous solution of sodium chloride. They are then dried over anhydrous magnesium sulfate, and concentrated on a rotary evaporator. The resulting white solid is recrystallized from a dichloromethane/methylcyclohexane mixture.

For this processing, the [1r]-2-(trans-2-isopropenyl-cis-5-methylcyclohexyl)acetyl chloride was prepared beforehand according to Corey E. J. and Carpino P. (Tet. Lett., 31, 3857, 1990).

Mp=104° C.; yield 80%.

¹H NMR: CDCl₃, 200 MHz: 0.86 (d, 6 Hz, 3H); from 0.60 to 1.05 (m, 2H); 1.65 (s, 3H); from 1.20 to 2.0 (m, 8H); 2.38 (d*d, 14/2 Hz, 1H); 3.87 (s, 3H); 4.35 (m, 2H); 4.70 (m, 2H); 5.70 (m, 2H); 6.80 (m, 3H).

¹³C NMR: CDCl₃, 50 MHz: 18.79; 22.57; 32.12; 32.34; 34.91; 36.69; 41.09; 41.97; 43.49; 51.74; 55.90; 110.66; 111.55; 114.34; 120.80; 130.44; 145.11; 146.71; 148.73; 172.53 ppm.

IR: σ(cm⁻¹) 3315, 2915, 2842, 1631, 1595, 1558, 1514, 1452, 1427, 1274, 1247, 1232, 1210, 1177, 1156, 1123, 1039, 886, 847, 828, 794, 751, 737.

### EXAMPLE 15

### Preparation of Compound 8

Compound 8 is obtained according to scheme 4 starting from compound III-1:

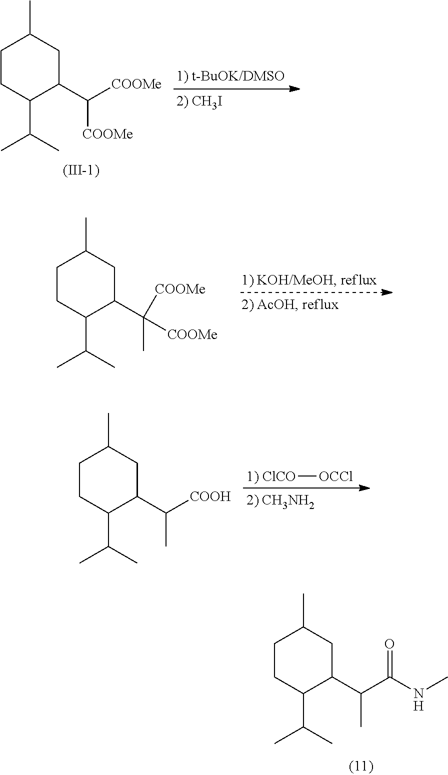

20 g (0.074 mol) of malonic ester III-1 and then 0.1 l of anhydrous dimethyl sulfoxide are introduced into a conventional apparatus. The resulting solution is cooled to between 10 and 15° C. (crystallization limit). 8.75 g (0.078 mol, 1.05 eq.) of potassium t-butoxide are slowly introduced such that the temperature of the medium does not exceed 20° C. The solution obtained is kept at ambient temperature for 2 hours and then 12.6 g (0.089 mol, 1.2 eq.) of iodomethane are added dropwise.

The mixture is stirred for 2 hours and is then poured into 0.3 l of clear water. The aqueous phase is extracted with four times 0.1 l of methylcyclohexane. The combined organic phases are washed with three times 0.1 l of clear water, dried over magnesium sulfate, and then concentrated. The resulting white solid is recrystallized from methylcyclohexane at −30° C. 17.24 g of dimethyl ester of [1r]-2-(trans-2-isopropyl-cis-5-methylcyclohexyl)-2-methylmalonic acid are obtained, i.e. a yield of 82%.

8.4 g of KOH (0.15 mol, 1.5 eq.), 10 g of water, 0.2 l of methanol and then 15 g (0.05 mol) of the previously obtained ester are introduced successively into a conventional apparatus. The mixture is brought to reflux for 12 hours, and then the condenser is replaced with a distillation apparatus. The methanol is distilled off by replacing it with water as the distillation progresses. The distillation is interrupted when the temperature of the vapors reaches 80° C. The solution, cooled to ambient temperature, is extracted twice with 0.1 l of methyl t-butyl ether, and then cooled to 0° C. and acidified to pH=3. The suspension obtained is extracted three times with 0.2 l of methyl t-butyl ether. The organic phase is washed to neutrality, dried over magnesium sulfate, and then concentrated. The residual solid is recrystallized from a mixture of methyl t-butyl ether and methylcyclohexane. 10.9 g of [1r]-2-(trans-2-isopropyl-cis-5-methylcyclohexyl)-2-methylmalonic acid are isolated in the form of a white solid, i.e. a yield of 85%.

12 g of acetic acid and 0.5 g of 96% sulfuric acid are introduced successively into a conventional apparatus. The solution is stirred for 5 minutes and then 10.9 g (0.0425 mol) of the acid previously obtained are added. The mixture obtained is brought to reflux for 12 hours and is then cooled to ambient temperature and 1 g of sodium acetate trihydrate is added and the resulting solution is then stirred for 0.5 hour. The acetic acid is distilled off under vacuum and the residue is taken up in 0.2 l of water, which is subsequently extracted with three times 0.05 l of methyl t-butyl ether. After drying over magnesium sulfate, the solution is concentrated and the residue is distilled under vacuum. The [1r]-2-(trans-2-isopropyl-cis-5-methylcyclohexyl)propionic acid obtained in the form of a viscous oil is taken up in 0.05 l of anhydrous toluene.

10 g (0.04 mol) of the preceding acid in 0.05 l of anhydrous toluene and then 2 drops of N,N-dimethylformamide are introduced successively into a conventional apparatus. Freshly purified oxalyl chloride (6.45 g; 1.3 eq.) is poured into the dropping funnel and then added dropwise to the round-bottomed flask. The resulting mixture is stirred for 12 hours at ambient temperature and then the solvent is distilled off under partial vacuum. The residue is taken up in 0.025 l of anhydrous toluene, which is distilled off under vacuum. The residue is taken up with 0.025 l of anhydrous toluene.

0.025 l of a 40% solution of monomethylamine in water is introduced into a conventional apparatus, said solution being brought to 0° C. using a water/ice bath. The previous solution of acid chloride in toluene is introduced into the dropping funnel and then added dropwise to the round-bottomed flask. The resulting mixture is stirred for 2 hours and then the phases are separated. The aqueous phase is retained. The organic phase is washed twice with 0.05 l of clear water, 0.05 l of 5% hydrochloric acid and then again with 0.1 l of clear water. This organic phase is dried over magnesium sulfate and then concentrated. The residual solid is recrystallized from a mixture of methyl t-butyl ether and methylcyclohexane so as to give 8.3 g of compound 8 in the form of a mixture of two stereoisomers (in a 2:1 proportion).

Mp=133° C.; yield 74%.

The product consists of two stereoisomers, which will be denoted respectively Major and minor when the attribution of the NMR signals is unambiguous.

¹H NMR: CDCl₃, 200 MHz: 0.72 (d, 6.8 Hz, 3H); 0.88 (d, 7.0 Hz, 3H); 0.91 (d, 7.0 Hz, 3H); from 0.60 to 1.00 (m, 4H); 1.14 (d, 7.2 Hz, 3H); from 1.00 to 1.50 (m, 2H); from 1.60 to 1.90 (m, 3H); 2.10 (m, 1H); 2.59 (q.d. 2.6/7.2 Hz, 1H); 2.79 (d, 4.8 Hz, 3H M); 2.82 (d, 4.8 Hz, 3H m); 5.50 (s, 1H).

¹³C NMR: CDCl₃, 50 MHz: 15.63 (M); 15.81 (m); 21.91 (m); 22.05 (M); 23.08 (m); 23.12 (M); 24.70 (m); 24.84 (M); 24.70 (m); 24.84 (M); 26.52 (M); 26.58 (m); 26.74 (m); 27.24 (M); 33.10 (m); 33.61 (M); 35.35 (M); 35.68 (m); 35.82 (m); 37.31 (M); 40.35 (m); 40.79 (M); 42.22 (m); 43.97 (m); 44.29 (m); 44.83 (M); 175.80 (M); 177.10 (m) ppm.

MS: m/z=225 [M+.]; 210; 194; 182; 154; 140; 123; 100; 87(100); 69 amu.

IR: σ(cm⁻¹) 3334, 3070, 2950, 2866, 1644, 1547, 1455, 1407, 1368, 1298, 1243, 1159, 1025, 685.

### EXAMPLE 16

### Preparation of Compound 9

According to the protocol of example 15, replacing the iodomethane with 9.70 g (0.089 mol; 1.2 eq.) of bromoethane, 15.9 g of dimethyl ester of [1r]-2-(trans-2-isopropyl-cis-5-methylcyclohexyl)-2-ethylmalonic acid are obtained, i.e. a yield of 72%, and then 8.13 g of compound 9 in the form of a mixture of two stereoisomers (in a 1:1 proportion) are obtained.

Mp=137° C.; yield 68%.

¹H NMR: CDCl₃, 200 MHz: from 0.70 to 1.00 (m, 12H); from 1.00 to 2.10 (m, 12H); 2.31 (d.t, 2.5/12 Hz, 1H); 2.80 (d, 4.9 Hz, 3H); 2.83 (d, 4.9 Hz, 3H); 5.60 (s, 1H).

¹³C NMR: CDCl₃, 50 MHz: 13.13; 13.75; 15.46; 15.70; 17.79; 21.90; 22.00; 23.05; 23.15; 24.34; 24.81; 24.89; 26.40; 26.66; 26.85; 27.18; 33.33; 33.67; 35.26; 35.69; 36.48; 36.68; 42.54; 43.41; 44.44; 44.76; 48.98; 49.19; 175.14; 176.29 ppm.

MS: m/z=239 [M+.]; 224; 210; 196; 180; 168; 154; 101(100); 95; 86; 81 amu.

IR: σ(cm⁻¹) 3298, 2958, 2870, 1644, 1547, 1456, 1409, 1365, 1239, 1159, 800, 685.

### EXAMPLE 17

### Preparation of Compound 15

Compound 15 is obtained according to scheme 3 starting from the compound III-2:

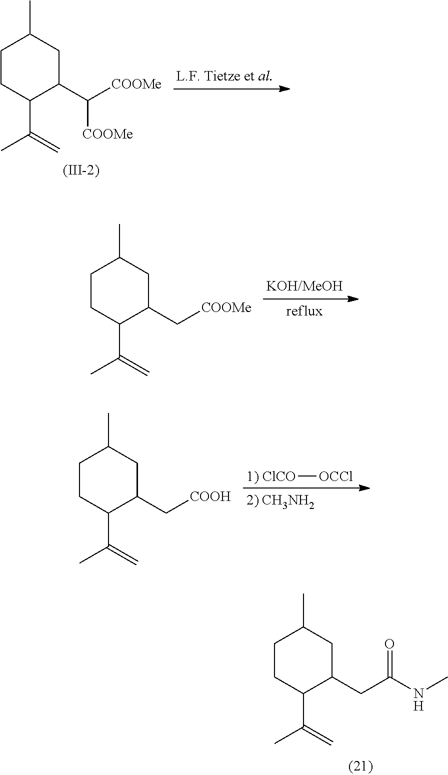

The experimental conditions are identical to those described by L. F. Tietze et al.

Mp=116° C.; yield 83%.

¹H NMR: CDCl₃ 200 MHz: 0.87 (d, 6.4 Hz, 3H); from 0.50 to 1.10 (m, 2H); 1.66 (s, 3H); from 1.30 to 2.00 (m, 8H); 2.33 (d*d, 2.8/13.8 Hz, 1H); 2.78 (d, 4.8 Hz, 3H); 4.71 (s, 2H); 5.83 (s, 1H).

¹³C NMR: CDCl₃, 50 MHz: 19.18; 22.96; 26.56; 32.55; 32.73; 35.53; 37.01; 41.46; 42.16; 52.16; 111.81; 149.15; 173.90 ppm.

MS: m/z=209 [M^{+.}]; 194; 166; 136; 121; 109; 95; 73 (100) amu.

IR: σ(cm⁻¹) 3252, 3082, 2923, 2842, 1642, 1570, 1433, 1375, 1261, 1183, 1155, 899, 768, 623.

¹³C NMR: CDCl₃, 50 MHz: 20.37; 20.46; 23.76 (2C); 27.30; 27.49; 33.80 (2C); 33.87; 33.97; 35.79; 35.94; 37.90; 38.26; 42.51; 43.26; 50.28; 50.86; 52.96; 53.33; 54.23; 55.66; 113.47; 113.82; 148.89; 149.42; 169.24; 170.72; 174.17; 174.28 ppm.

MS: m/z=267 [M^{+.}]; 252; 236; 221; 208; 184; 144; 136; 131(100); 121; 107; 101; 93; 79 amu.

IR: σ(cm⁻¹) 3265, 3100, 2923, 2860, 1747, 1642, 1568, 1437, 1330, 1407, 1300, 1265, 1155, 1119, 897, 758, 728.

### EXAMPLE 18

### Preparation of Compound VIa

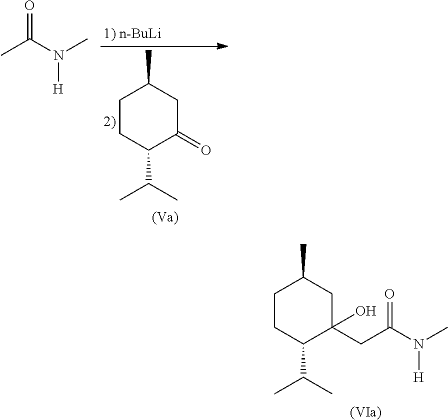

0.4 l of methyl t-butyl ether and then 0.16 l (2.5N, 0.4 mol) of n-butyllithium in hexane are introduced successively into a conventional apparatus. The mixture obtained is brought to 0° C. and then a solution of 14.6 g (0.2 mol) of N-methylacetamide in 0.1 l of methyl t-butyl ether is added slowly by the dropping funnel in such a way as to maintain the temperature at 0° C. The resulting suspension is stirred for 2 hours at the same temperature and then for 18 hours at ambient temperature, so as to give a pale yellow solution. The latter is brought to −20° C. and then 30.8 g (0.2 mol) of (L)-menthone (Va) are added dropwise. The reaction mixture is kept at ambient temperature for 18 hours before being hydrolyzed. The crude product obtained after concentration is distilled under high vacuum in order to eliminate the menthone present (Bp=35-40° C./26.6 Pa) without exceeding 60° C. in the round-bottomed flask. The residue is recrystallized twice from hexane at −30° C. in order to obtain a pure product. 30.5 g of compound VIa are isolated in the form of a white solid.

Mp=81° C.; yield 67%.

¹H NMR: CDCl₃, 200 MHz: 0.85 (d, 6.2 Hz, 3H); 0.89 (d, 7.0 Hz, 3H); 0.93 (d, 7.0 Hz, 3H); from 0.70 to 1.10 (m, 3H); from 1.40 to 1.60 (m, 2H); from 1.60 to 1.90 (m, 3H); 1.96 (d, 14.3 Hz, 1H); from 1.90 to 2.10 (m, 1H); 2.78 (d, 14.3 Hz, 1H); 2.82 (d, 4.6 Hz, 3H); 3.97 (s, 1H); 6.29 (s, 1H).

¹³C NMR: CDCl₃, 50 MHz: 16.28; 18.84; 20.65; 22.02; 24.40; 24.68; 26.12; 33.47; 44.01; 45.19; 49.02; 72.25; 172.38 ppm.

MS: m/z=227 [M^{+.}]; 212; 210; 209; 194; 170; 157; 142; 115; 109; 100; 95; 86; 81; 73 (100) amu.

IR: σ(cm⁻¹) 3311, 2950, 2867, 1640, 1557, 1451, 1407, 1367, 1346, 1239, 1183, 1159, 1071, 1052, 987, 889, 821, 713, 654.

### EXAMPLE 19

### Preparation of Compound 17

Compound 17 is prepared via the compound VIa obtained in example 18 according to scheme 5:

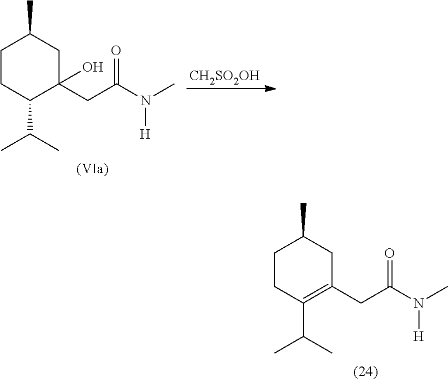

11.35 g (0.05 mol) of compound VIa, 0.1 l of anhydrous toluene and then 2 drops of methanesulfonic acid are introduced successively into a conventional apparatus. The resulting solution is stirred for 16 hours at reflux and is then cooled and neutralized with 0.05 l of a 1N aqueous solution of sodium bicarbonate. After drying and concentration under vacuum, the residue is analyzed by gas chromatography; several isomers are distinguishable, with the major isomer accounting for more than 75% of the mixture. This residue is recrystallized from hexane at −30° C., two times in a row, so as to give compound 17 in the form of a white solid.

Mp=82° C.; yield 64%.

¹H NMR: CDCl₃, 200 MHz: 0.87 (d, 6.4 Hz, 3H); 0.90 (d, 6.3 Hz, 6H); from 0.60 to 1.20 (m, 2H); from 1.40 to 2.00 (m, 6H); from 2.40 to 2.60 (m, 1H); 2.72 (d, 4.8 Hz, 3H); 2.81 (d, 16.5 Hz, 1H); 2.97 (d, 12.5 Hz, 1H); 5.95 (s, 1H).

¹³C NMR: CDCl₃, 50 MHz: 20.65; 21.10; 21.96; 23.53; 26.66; 29.38; 29.90; 31.48; 40.07; 41.09; 123.14; 140.84; 172.14 ppm.

MS: m/z=209 [M^{+.}]; 194; 166; 136; 121; 109; 95; 81; 73 (100) amu.

IR: σ(cm⁻¹) 3288, 3079, 2960, 2872, 1650, 1549, 1458, 1403, 1338, 1260, 1160, 1052, 716, 681.

### EXAMPLE 20

### Preparation of Compound 18

Compound 18 is prepared via the compound VIa obtained in example 18 according to scheme 5:

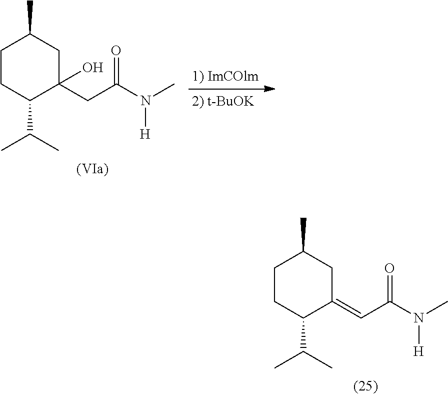

11.35 g (0.05 mol) of compound VIa, 9.75 g (0.06 mol, 1.2 eq.) of N,N-carbonyldiimidazole and 0.1 l of anhydrous tetrahydrofuran are introduced successively into a conventional apparatus. The solution thus obtained is stirred for 2 hours at ambient temperature and then for 2 hours at 50° C. The solution is cooled to ambient temperature and then a solution of 7.84 g (0.07 mol, 1.4 eq.) of potassium t-butoxide in 0.1 l of anhydrous THF is added without exceeding 30° C. in the reaction medium. After 18 hours at ambient temperature, the medium is hydrolyzed, dried and concentrated under vacuum.

An analysis by vapor-phase chromatography of the concentrate shows the presence of 4 isomers in respective proportions of 9:6:14:71%.

The major isomer, compound 18, is isolated, by crystallization from hexane at −30° C., with a yield of 58%, i.e. 6.06 g of a white solid.

Mp=64° C.

¹H NMR: CDCl₃, 200 MHz: 0.86 (d, 6.2 Hz, 3H); 0.89 (d, 6.6 Hz, 3H); 0.95 (d, 6.6 Hz, 3H); from 1.00 to 1.50 (m, 2H); from 1.60 to 2.00 (m, 5H); from 2.30 to 2.50 (m, 1H); 2.82 (d, 4.8 Hz, 3H); 3.09 (d*d, 4.1/12.9 Hz, 1H); 5.56 (s, 1H); 6.04 (s, 1H).

¹³C NMR: CDCl₃, 50 MHz: 19.52; 20.70; 21.96; 26.01; 26.78; 27.37; 31.89; 33.42; 36.12; 51.98; 115.97; 157.98; 168.40 ppm.

MS: m/z=209 [M^{+.}]; 194; 167; 152; 137; 121; 109; 101; 95; 81; 73 (100) amu.

IR: σ(cm⁻¹) 3273, 3083, 2950, 2866, 1655, 1627, 1559, 1443, 1408, 1385, 1263, 1244, 1195, 1160, 895, 873, 745, 678.

### EXAMPLE 21

### Preparation of the N-methylamide of (1′S,2′S,5′S)-2-[(2′-isopropenyl-5′-methyl)cyclohex-1-yl]acetic acid (compound 1′)

Compound 1′, the (1S,2S,5S) enantiomer of compound 1, is obtained according to a protocol identical to examples 1 and 2 starting from (S)-citronellal.

Mp=105° C.; [α]_{D}²⁵=+76.4° (c=1; EtOH).

### EXAMPLE 22

### Preparation of the N-methylamide of (1′R,2′R,5′R)-2-[(2′-isopropenyl-5′-methyl)cyclohex-1-yl]acetic acid (compound 1″)

Compound 1″, the (1R,2R,5R) enantiomer of compound 1, is obtained according to a protocol identical to examples 1 and 2 starting from (R)-citronellal.

Mp=105° C.; [α]_{D}²⁵=−76.4° (c=1; EtOH).

### EXAMPLE 23

### Comparison of the Enantiomers 1′ and 1″

The enantiopure compounds 1′ and 1″ obtained in examples 21 and 22 and also the racemic mixture of compound 1 obtained in example 2 were subjected to a panel of flavor experts for a comparative evaluation of the organoleptic and physiological qualities.

This evaluation did not make it possible to demonstrate any notable difference between the two enantiomers 1′ and 1″ taken separately, nor with the racemic mixture, both in terms of the perception threshold and in terms of the intensity of the refreshing effect felt.

### EXAMPLE 24

### Results of Evaluation of Compound 1

Food Flavoring

The molecule was evaluated in mineral water at a dosage of 30 ppm. The panelists felt a freshness which came very rapidly after swallowing and described the aromatic note of the drink: minty, pine, liquoricy, warming, piquant, salivating.

The molecule was tested in a mint flavoring and evaluated in fondant at a dosage of 50 ppm. The panelists perceived a feeling of intense freshness which lasts for a few minutes.

The molecule was at a dosage of 10% in a mint flavoring, the flavoring obtained was at a dose of 0.2% in sweet confectionery.

| | Formula A | Formula B |
|---|---|---|
| Eucalyptol | 3% | 3% |
| Spearmint essence | 7% | 7% |
| Natural menthol | 50% | 40% |
| Compound 1 | 0% | 10% |
| China mint essence | 40% | 40% |

The panel of tasters perceived freshness on impact and for several minutes. The freshness of the flavoring not containing compound 1 (formula A) was not as intense and had a shorter period in the mouth than the freshness perceived with the flavoring containing compound 1 (formula B).

The molecule was tested at 10% in a citrus fruit flavoring, the flavoring obtained was at a dose of 0.3% in fatty filling.

The panelists perceived a persistent freshness, and a change in the lemon profile. The product not contained in compound 1 did not show any particular freshness.

Toothpaste Flavoring

These compositions are strawberry flavorings for children that do not contain mint, which should make it possible to demonstrate the freshness.

| | Composition A | Composition B |
|---|---|---|
| | (%) | (%) |
| Hedione at 1% in PG | 0.1 | 0.1 |
| C16 aldehyde at 1% in PG | 0.5 | 0.5 |
| cis-3-Hexenyl acetate | 0.1 | 0.1 |
| Hexyl acetate | 0.4 | 0.4 |
| Methyl cinnamate | 0.9 | 0.9 |
| cis-3-Hexenol | 1.8 | 1.8 |
| Isoamyl acetate | 0.6 | 0.6 |
| Ethyl 2-methylbutyrate | 1.5 | 1.5 |
| Ethyl butyrate | 5 | 5 |
| Ethyl vanillin | 4 | 4 |
| Ethyl caproate | 0.7 | 0.7 |
| Amyl butyrate | 1 | 1 |
| Strawberry furanone | 0.9 | 0.9 |
| Butyric acid | 0.3 | 0.3 |
| Maltol | 1 | 1 |
| Vanillin | 0.1 | 0.1 |
| Isoamyl butyrate | 0.1 | 0.1 |
| Gamma-undecalactone | 0.1 | 0.1 |
| Propylene glycol | 80.9 | 60.9 |
| Compound 1 | 0 | 20 |

These two flavorings were applied in a gel at a dosage of 1% and were tested with brushing.

STRAWBERRY A: strawberry, lightly baked green note/vanilla-flavored

STRAWBERRY B: strawberry, slight freshness perceived quite rapidly, green note, light, more vanilla-flavored.

### EXAMPLE 25

### Results of Evaluation of Compound 10

Food Flavoring

The evaluation of the organoleptic properties of compound 10 was carried out in mineral water at a concentration of 1 ppm. The flavor experts felt a burning and salivation sensation which comes immediately after depositing on the tongue.

Compound 10 was subsequently tested in a curry/capsicum flavoring and evaluated in crackers at 5% (final dosage: 500 ppm).

| | Formula A | Formula B |
|---|---|---|
| | (%) | (%) |
| Chinese cinnamon essence | 0.15 | 0.15 |
| Cinnamic aldehyde of cinnamon | 0.35 | 0.35 |
| Natural methylcyclopentenolone | 0.45 | 0.45 |
| hydrate | | |
| Nutmeg essence | 0.7 | 0.7 |
| Cumin essence | 0.8 | 0.8 |
| Capsicum ext. NPU | 0.15 | 0.15 |
| Indian ginger essence | 0.15 | 0.15 |
| Clove essence | 0.15 | 0.15 |
| Celery essence | 0.35 | 0.35 |
| Anethol B21 1% Elsun | 1.5 | 1.5 |
| 2% powdered curcuma | 15 | 15 |
| Cayenne pepper oleo resin | 1.5 | 0 |
| Compound 10 | 0 | 5 |
| Non-oiled yeast extract 2006 | 4 | 4 |
| powder | | |
| Nonionized powdered garlic | 7 | 7 |
| Extrafine sodium glutamate | 15 | 15 |
| Fine salt 170 microns | 17 | 17 |
| Maltodextrin | qs 100% | qs 100% |

The panelists found flavoring B to be as piquant as formula A. The aromatic note is slightly modified, compound 10 providing a more longlasting sensation.

Flavorings for Application in Oro-Dental Hygiene

Compound 10 was tested in various applications: in an alcoholic mouthwash and a nonalcoholic mouthwash and in toothpaste. The panel of tasters felt a piquant and warming sensation. This molecule is very advantageous since it provides a tingling sensation on the edge of the tongue, which is piquant like a capsicum, and salivating.

| | Reference | | | |
|---|---|---|---|---|
| | flavoring | Flavoring A | Flavoring B | Flavoring C |
| Idaho mint | 45 | 45 | 45 | 45 |
| essence | | | | |
| Lime essence | 45 | 45 | 45 | 45 |
| Citronella | 6 | 6 | 6 | 6 |
| essence | | | | |
| Cinnamon | 1 | 1 | 1 | 1 |
| essence | | | | |
| Orange | 2 | 2 | 2 | 2 |
| essence | | | | |
| Lime | 38 | 38 | 38 | 38 |
| terpenes | | | | |
| Anethol | 40 | 40 | 40 | 40 |
| Lemon | 75 | 75 | 75 | 75 |
| essence | | | | |
| Citral | 20 | 20 | 20 | 20 |
| Eucalyptol | 45 | 45 | 45 | 45 |
| Deterpenated | 150 | 150 | 150 | 150 |
| Chinese mint | | | | |
| essence | | | | |
| Menthol | 375 | 375 | 375 | 375 |
| Physcool | 30 | 30 | 30 | 30 |
| Triacetin | 128 | 113 | 108 | 122 |
| Compound 10 | 0 | 15 | 20 | 6 |

Mouthwash (without Alcohol):

Dosage of flavoring 0.2%

Reference flavoring: lemony, cola, cool, minty

Flavoring B: lemony, piquant, cola, cool, minty, piquant sensation on the end of the tongue

Mouthwash (with 6% Alcohol):

Dosage of flavoring 0.2%

Reference flavoring: lemony, cola, cool, minty

Flavoring A: lemony, piquant, cola, cool, minty, piquant sensation on the end of the tongue

In Toothpaste, Silica Base:

Dosage of flavoring 1%

Reference flavoring: lemony, cola, cool, minty

Flavoring C: lemony, piquant, cola, cool, minty, piquant sensation on the end of the tongue.

1. A compound of general formula (I): 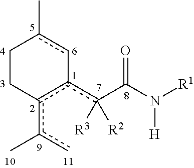
in which:
each of the dashed bonds is independently present or absent, provided that two successive dashed bonds are not simultaneously present;
R¹, which is other than an ethyl, is chosen from a C₁-C₆ alkyl, a C₂-C₆ alkenyl, a C₃-C₆ cycloalkyl and a C₅-C₆ cycloalkenyl; it being possible for each of these groups to be optionally substituted with at least one hydroxyl function, carboxyl function, nitrile function, C₁-C₆ alkoxy function, C₁-C₆ alkoxycarbonyl function, C₁-C₄ cyanoalkyl function and benzo[1,3]dioxol-5-yl function;
if the dashed bond between the atoms C1 and C7 is absent,
R² and R³ are independently a hydrogen atom, or a C₁-C₆ alkyl;
if the dashed bond between the atoms C1 and C7 is present,
R² is a hydrogen atom, or a C₁-C₆ alkyl; and
R₃ is absent.

2. The compound as claimed in claim 1, characterized in that R¹ is a methyl.

3. The compound as claimed in claim 2, characterized in that R³ is a hydrogen atom and R² is chosen from a hydrogen atom and a methyl or ethyl group.

4. The compound as claimed in claim 2, characterized in that R² and R³ are two hydrogen atoms.

5. The compound as claimed in claim 2, characterized in that said compound is chosen from:
N-methyl-2-(2-isopropyl-5-methylcyclohexyl)acetamide
2-(6-isopropyl-3-methylcyclohex-2-enyl)-N-methylacetamide
2-(2-isopropenyl-5-methylcyclohexyl)-N-methylacetamide
2-(2-isopropyl-5-methylcyclohex-1-enyl)-N-methylacetamide
2-(2-isopropyl-5-methylcyclohexylidene)-N-methylacetamide
2-(2-isopropyl-5-methylcyclohexyl)-N-methylpropionamide
3-(2-isopropyl-5-methylcyclohexyl)-N-methylbutyramide.

6. The compound as claimed in claim 1, characterized in that R¹ has a steric hindrance that is greater than that of an ethyl radical.

7. The compound as claimed in claim 6, characterized in that R¹ is a phenyl or benzyl group substituted with at least one hydroxyl, carboxyl, nitrile, C₁-C₄ alkoxy, C₁-C₄ alkoxycarbonyl or C₁-C₄ cyanoalkyl function.

8. The compound as claimed in claim 7, characterized in that R¹ is chosen from a 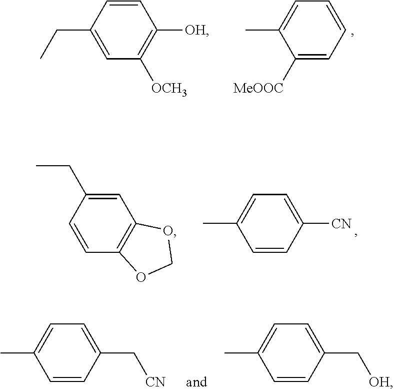
group.

9. The compound as claimed in claim 6, characterized in that R¹ is chosen from an isopropyl, isobutyl, —CH₂COOH, —CH₂COOMe, —CH₂COOEt, —CH₂OH, —CH₂CH₂OH and —CH₂C(CH₃)₂OH group.

10. The compound as claimed in claim 6, characterized in that R² and R³ are two hydrogen atoms.

11. The compound as claimed in claim 6, characterized in that said compound is chosen from:
N-(4-hydroxy-3-methoxybenzyl)-2-(2-isopropyl-5-methylcyclohexyl)acetamide
N-(4-hydroxy-3-methoxybenzyl)-2-(2-isopropenyl-5-methylcyclohexyl)acetamide
N-benzo[1,3]dioxol-5-ylmethyl-2-(2-isopropyl-5-methylcyclohexyl)acetamide
[2-(2-isopropyl-5-methylcyclohexyl)acetylamino]ethyl acetate
[2-(2-isopropyl-5-methylcyclohexyl)acetylamino]methyl acetate
N-(2-hydroxy-2-methylpropyl)-2-(2-isopropyl-5-methylcyclohexyl)acetamide
N-(2-hydroxyethyl)-2-(2-isopropyl-5-methylcyclohexyl)acetamide
N-(4-cyanomethylphenyl)-2-(2-isopropyl-5-methylcyclohexyl)acetamide
N-(4-cyanophenyl)-2-(2-isopropyl-5-methylcyclohexyl)acetamide
[2-(2-isopropyl-5-methylcyclohexyl)acetylamino]acetatic acid
N-isobutyl-2-(2-isopropyl-5-methylcyclohexyl)acetamide
N-(2-methyloxycarbonylphenyl)-2-(2-isopropyl-5-methylcyclohexyl)acetamide.

12. An aromatic composition comprising at least one compound of formula (I) as claimed in claim 1.

13. An aromatic composition of the food product type comprising at least one compound of formula (I) as claimed in claim 1.

14. An aromatic composition which is part of the composition of a medicinal or paramedical product, suitable for intraoral application, comprising at least one compound of formula (I) as claimed in claim 1.

15. A cosmetic composition for caring for the face, the body or a part of the body, comprising at least one compound of formula (I) as claimed in claim 1.
